(19) United States
(12) Patent Application Publication
(10) Pub. No.: US 2003/0078471 A1
(43) Pub. Date: Apr. 24, 2003

(54) Manipulation of an organ

(76) Inventors: Frederick J. Foley, Bedford, NH (US); Thomas G. Adelman, West Baldwin, ME (US)

Correspondence Address: SHUMAKER & SIEFFERT, P. A., 8425 SEASONS PARKWAY, SUITE 105, ST. PAUL, MN, 55125, US

(21) Appl. No.: 10/161,477

(22) Filed: May 31, 2002

Related U.S. Application Data
(60) Provisional application No. 60/336,345, filed on Oct. 18, 2001.

(51) Int. Cl. A61F 2/00

(52) U.S. Cl. 600/37

(57) ABSTRACT

Devices for and methods of moving, manipulating, or holding an organ of the body, particularly a heart, are disclosed. A manipulating device includes a seal member, which includes two or more protrusions, that adheres to the surface of the organ when vacuum pressure is applied to a chamber defined by an inner surface of the seal member. The seal member may be made of a substantially compliant and flexible material. The protrusions may enable the seal member to more easily conform to the irregular shape of the organ, may accommodate patient-to-patient variations in the size and shape of organs, may increase the strength of the attachment between the seal member and the organ, may reduce the likelihood of damage to the surface of the organ and may expose areas of the organ for surgical procedures.

This application claims priority from U.S. Provisional Application Serial No. 60/336,345, filed Oct. 18, 2001, the entire content of which is incorporated herein by reference.

### TECHNICAL FIELD

The invention relates to devices capable of providing adherence to organs of the body for purposes of medical diagnosis and treatment. More particularly, the invention relates to devices capable of adhering to, and moving, manipulating, or holding an organ of the body, particularly a heart, for purposes of medical diagnosis and treatment.

### BACKGROUND

In many areas of surgical practice, it may be desirable to manipulate an internal organ without causing damage to that organ. In some circumstances, a surgeon may wish to turn, lift, or otherwise re-orient the organ so that surgery can be performed upon it. In other circumstances, a surgeon may wish to move the organ out of the surgical field of view. In still other circumstances, a surgeon may need to both re-orient the organ and also hold the organ in the new position to facilitate physical access to a specific part of the organ not readily accessible in the previous position.

Manipulating or holding the organ with the hands may not be feasible, safe, or optimal to the surgical activities to be performed. Many organs are slippery and are difficult manipulate or hold with the hands. Moreover, the surgeon's hands may not be able to manipulate or hold the organ, and perform the procedure at the same time.

The hands of an assistant may be bulky, becoming an obstacle to the surgeon. The hands of an assistant may be a particular problem in situations where surgery is to be performed on the organ. Also, manual support of an organ over an extended period of time can be difficult due to fatigue. Manipulating or holding an organ with an instrument, on the other hand, may damage the organ, especially if the organ is unduly squeezed, pinched, or stretched. Further, holding an organ improperly may also adversely affect the functioning of the organ.

The heart is an organ that can be more effectively treated in some surgical procedures if it can be manipulated. Typically, a patient's chest is opened via a sternotomy, the ribs pushed aside with a retractor, and the pericardial sac opened to expose the heart. In this presentation, the surgeon has primary access only to the anterior surface of the heart. In the case of treating cardiovascular disease by repairing partially or completely occluded coronary arteries, it is necessary to repair arteries located not only on the anterior surface of the heart, but also on the posterior and/or lateral surfaces of the heart. This procedure is referred to as cardiac artery bypass grafting (CABG).

CABG may be performed by surgeons upon hearts using the procedure of cardiopulmonary bypass (CPB), in which the beating of the heart is stopped by physiologic means and the patient's blood is circulated away from the heart and lungs and into a heart-lung machine for oxygenation. Although manipulating and holding the heart are still problematic using CPB, the procedure may permit the surgeon to manually manipulate the stopped heart using hands, portions of the pericardial sac, and sutures, permitting access to posterior, lateral and anterior heart surfaces. CPB, however, entails trauma to the patient, with attendant side effects and risks.

Recently, surgeons have developed methods to perform CABG on the beating heart, without CPB. CABG on a beating heart, referred to as “off pump” or “beating heart” surgery, offers reduced morbidity to the patient, but presents increased technical challenges to the surgeon, who must now lift and manipulate a beating heart to access the posterior and lateral surfaces without interfering with the effective normal output or hemodynamics of the heart. For example, merely lifting the beating heart with the hand can potentially cause partial obstruction of the blood flow out of the heart with a dangerous transient drop in blood pressure. Further, held insecurely, the heart may drop back into the chest, which may cause trauma to the heart, and may interfere with the progress of the operation.

While cardiothoracic surgeons have developed a variety of methods to manipulate the heart, the methods may be cumbersome, or may entail risk to the patient, or may not permit a sufficient operative field for the surgeon.

### SUMMARY

The invention presents an organ manipulation device for adhering to, and moving, manipulating, or holding an organ of the body. It should be noted that any references to “adhesion” or related terms do not use the term as it is frequently used in medicine, namely to describe an abnormal union of an organ or part with some other part by formation of fibrous tissue. Rather “adhesion” and related words refer to adherence, the process or one thing holding fast to another, without them becoming pathologically joined.

The organ manipulation device of the present invention provides for secure attachment to the organ, minimally interferes with the surgical field, and does not require the use of the surgeon's or assistant's hand. In addition, the device does not cause irreparable damage to the surface of the organ such as abrasion, laceration, or perforation. Some embodiments of the invention provide a device for and a method of repositioning the beating heart that enable the surgeon to lift and hold the heart without interfering with its hemodynamic function.

One embodiment of the organ manipulation device of the present invention comprises a seal member that contacts the surface of an organ. The seal member includes an inner surface that defines a chamber when the seal member is in contact with the surface of the organ. The seal member also includes a central body and at least two protrusions that extend outward from the central body. The protrusions may be substantially flexible and compliant.

The seal member may also include a distal edge that extends around the perimeter of the seal member and contacts the surface of the organ. The device may also include a coating located on the distal edge. The coating may be tacky.

In another embodiment, the organ manipulation device of the present invention comprises a seal member that contacts the surface of an organ. The seal member includes a central member, a skirt-like member, and at least two protrusions that extend outward from a central portion of the seal member. The skirt-like member may be substantially compliant and tacky. The central member may be less deformable than the skirt-like member, thereby imparting structural integrity to the seal member.

The invention also presents a method of adhering the organ manipulation device to an organ and moving, manipulating or holding the organ. One embodiment of the method comprises bringing a seal member into contact with a surface of an organ to define a chamber. The seal member has at least two protrusions. The method further comprises bringing the two protrusions into contact with the surface of the organ and applying vacuum pressure to the chamber to adhere the seal member to the surface of the organ.

The organ may be manipulated by manipulating the seal member. The organ may be manipulated by manipulating a handle attached to the seal member. Additionally, the organ may be held in a position by attaching the seal member to a support. If the organ is a heart, pacing pulses may be delivered to the heart.

Embodiments of the present invention may offer a number of advantages. For example, the protrusions may enable the seal member to more easily conform to the irregular shape of the organ, which may aid the formation of a good seal between the distal edge and the surface of the organ. In addition, the protrusions may access sites on the organ that are most conducive to the forming a good seal with the distal edge and may accommodate patient-to-patient variations in the size and shape of organs. Further, the protrusions may increase the surface area of the seal between the distal edge and the surface of the organ, which may increase the strength of the attachment between the seal member and the organ and may reduce the likelihood of damage to the surface of the organ, such as abrasion, laceration, or perforation. The protrusions may also expose areas of the organ for surgical procedures that require access to these areas of the organ.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a profile view of one embodiment of an organ manipulation device.

FIG. 2 is a bottom view of the seal member of the organ manipulation device shown in FIG. 1.

FIG. 3 is a cross-sectional view of a protrusion of the organ manipulation device, the cross-section taken at line A indicated in FIGS. 1 and 2.

FIG. 4 is a cross-sectional view of a protrusion of the organ manipulation device, the cross-section taken at line B indicated in FIGS. 1 and 2.

FIG. 5 is an enlargement of area D in FIG. 4 showing the distal edge of the seal member.

FIG. 6 is a cross-sectional view of a nipple of the organ manipulation device, the cross-section taken at line C indicated in FIG. 1.

FIG. 7 is a perspective view of one method of deployment of the organ manipulation device shown in FIG. 1, with the seal member including three protrusions on the apex of a human heart.

FIG. 8 is a cross-sectional side view of an alternate embodiment of the organ manipulation device deployed on the apex of a human heart.

FIG. 9 is a perspective view of another alternate embodiment of the organ manipulation device deployed on the apex of a human heart.

### DETAILED DESCRIPTION

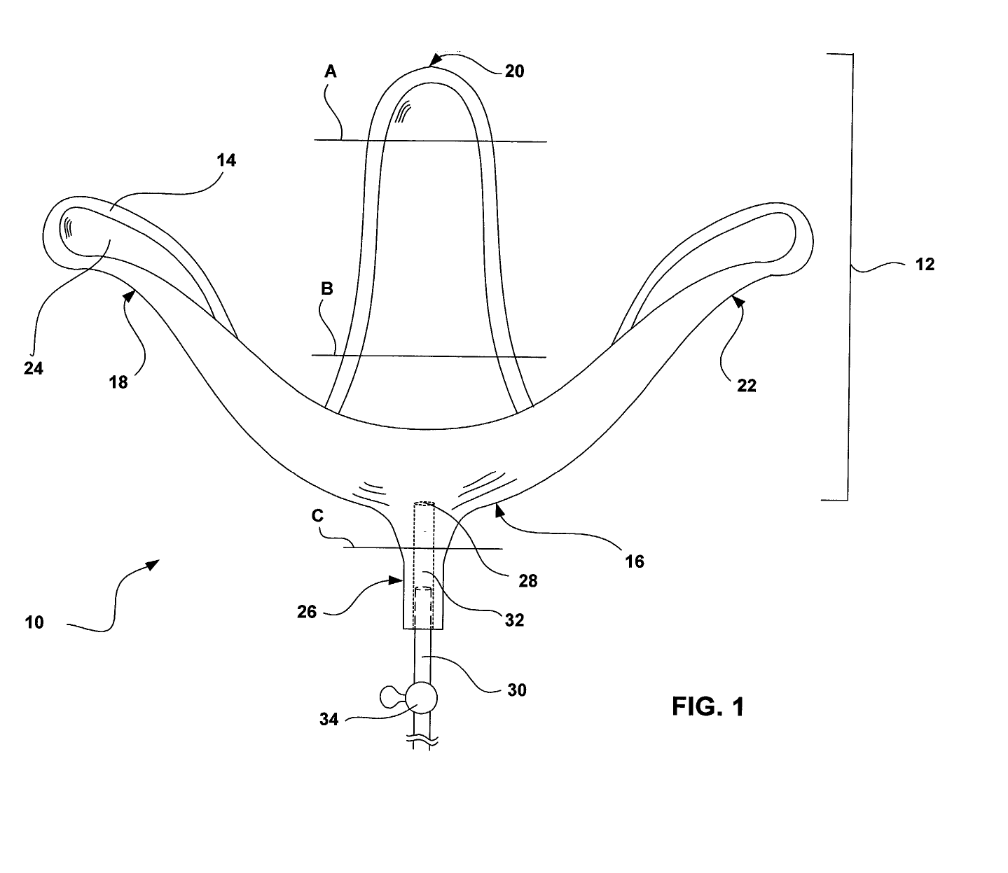

FIG. 1 is a profile view of one embodiment of the organ manipulation device 10 of the present invention. In this embodiment, organ manipulation device 10 includes a seal member 12 with a distal edge 14 that extends around the perimeter of seal member 12. Seal member 12 includes a central body 16 and three protrusions 18, 20 and 22. An inner surface 24 and distal edge 14 of seal member 12 cooperate to define a chamber when seal member 12 is in contact with the surface of an organ. Seal member 12 may include a vacuum port 28 in fluid communication with the chamber.

Manipulation device 10 may be coupled to a vacuum tube 30. Vacuum tube 30 may facilitate fluid communication between the chamber and a vacuum source (not shown). In some embodiments vacuum tube 30 may also serve as a support shaft for manipulating device 10. In these embodiments, vacuum tube must be made of a material of sufficient tensile strength to support the weight of an organ. In other embodiments, manipulating device 10 may be supported with a dedicated support shaft such as a plastic or metal shaft. In that case, vacuum tube 30 may provide little or no load-bearing capability. Instead, vacuum tube 30 may be disposed proximal to or within such a shaft. Vacuum tube 30 and/or the support shaft may be flexible.

The proximal portion of seal member 12 may be shaped into a hollow tube or nipple 26. Nipple 26 may define a passage 32, which is in fluid communication with vacuum port 28. Nipple 26 may be configured to receive vacuum tube 30 within passage 32. The invention is not limited to such a coupling, however. Vacuum tube 30 may be coupled to seal member 12 with any type of connector. The connector may be, for example, a fixed joint, a flexible joint or a swivel connection affixed to the proximal portion of seal member 12 that permits fluid communication between vacuum tube 30 and the chamber via vacuum port 28.

In addition to receiving vacuum tube 30, nipple 26 may also serve as a connection point between manipulation device 10 and a handle (not shown), which may further facilitate manipulation of the organ, or a fixed support (not shown), which may facilitate holding the organ in a position. Nipple 26 may be connected to a variety of handles or fixed supports. For example, a collar of rigid material, such as metal or polymer, can be attached to nipple 26 using adhesive. This rigid collar may facilitate connection to a handle or fixed support. Alternatively, the external diameter of nipple 26 could be sized and shaped so that it can be gripped by the clamp of a handle or support. Nipple 26 could also itself be used as a handle, allowing a surgeon or assistant to manipulate or hold the organ. As a further alternative, a handle or support could be attached to vacuum tube 30, a dedicated support shaft, or another location on seal member 12 instead of or in addition to nipple 26.

In embodiments that utilize a vacuum source, the vacuum source applies vacuum pressure to the chamber, which reduces the fluid pressure within the chamber and facilitates the adherence of seal member 12 to the organ. The vacuum source, may, for example, be a simple manual piston operated vacuum source, such as a syringe with appropriate valving. In other embodiments, the vacuum source may be a mechanical vacuum source, such as a rotary pump. The invention is not limited to use with a particular type of vacuum source.

Vacuum tube 30 may include a valve such as stopcock 34, to allow or prevent air from moving through vacuum tube 30. With stopcock 34 open, vacuum pressure may be applied to the chamber using a vacuum source. Vacuum pressure may be maintained by shutting stopcock 34. Alternatively, the organ may be moved into engagement with seal member 12 by the surgeon or assistant, thereby expelling air through open stopcock 34 and through vacuum tube 30. Closing stopcock 34 prevents air from entering seal member 12 via vacuum tube 30, and may create a partial vacuum or negative pressure in the chamber without the need for an applied vacuum. Stopcock 34 may also be used to release vacuum pressure, to allow the organ to disengage from seal member 12. Alternatively, a valve may be included in vacuum port 28 or nipple 26.

The difference between the pressure outside of the seal member 12 and inside the chamber forms a substantial seal between edge 14 of seal member 12 and the surface of the organ. Upon application of vacuum pressure, seal member 12, and particularly protrusions 18, 20 and 22, may deform slightly, conforming to the surface of the tissue and helping to form the seal. It is preferred that seal member 12 be flexible and compliant to substantially conform to the irregular surface of the organ such that the substantial seal may be formed. In embodiments where seal member 12 is to be adhered to a beating heart, it is further preferred that seal member 12 be flexible and compliant such that the substantial seal is not affected by the beating of the heart.

Thus, it is preferred that seal member 12 is made of one or more materials that exhibit adequate levels of flexibility and compliance. The materials may, for example, include elastomers such as silicone, natural rubber, synthetic rubber, and polyurethane, and more compliant materials, such as silicone gel, hydrogel, or closed cell foam. Seal member 12 in this embodiment includes a one-piece cast of silicone of sufficiently low durometer to permit deployment and sealing over the curved surfaces of organs, such as the curved surface of a heart, while maintaining sufficient structural integrity under vacuum pressure and the weight of an organ. The durometer of the silicone may, for example, be within the range from 5 to 50 Shore A.

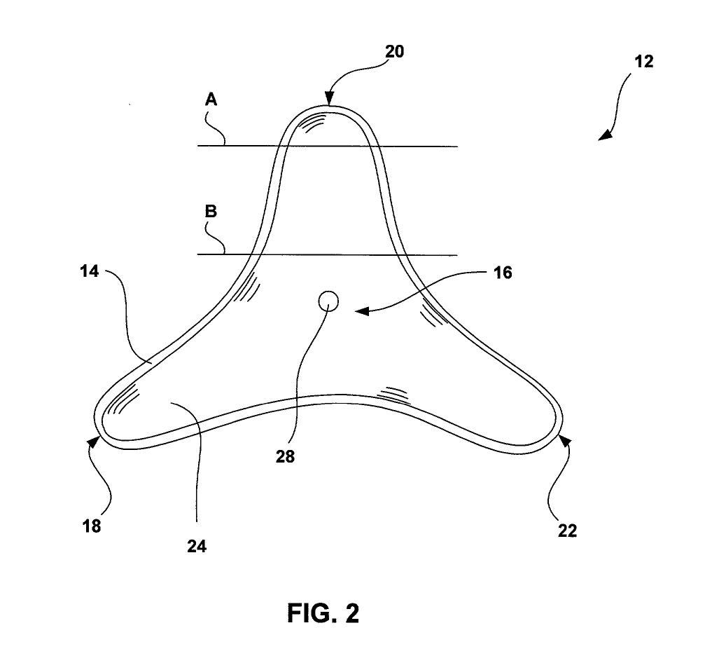

FIG. 2 is a bottom view of seal member 12. Edge 14, central body 16, protrusions 18, 20 and 22, inner surface 24, and vacuum port 28 are visible. While seal member 12 of FIGS. 1 and 2 includes three protrusions 18, 20 and 22, in other embodiments of device 10 there may be configurations of seal member 12 with two protrusions or more than three protrusions. As shown in FIG. 2, protrusions 18, 20 and 22 are substantially equidistant from each other, and of equal length and width. Further, in this embodiment, each of protrusions 18, 20, and 22 exhibits a long and narrow geometry, with its length two to six times its width, and tapers slightly toward its tip.

The configuration of protrusions 18, 20 and 22 depicted in FIGS. 1 and 2 has been demonstrated to provide advantages with respect to the attachment of seal member 12 to the irregular and asymmetric shape of a peri-apical site of a heart. This configuration could, however, also accommodate irregular shapes of other organs, such as the kidneys or the stomach. Moreover, the number or protrusions, the angle or distance between protrusions, and the length, width and shape of each individual protrusion may be varied to accommodate a variety of organs and attachments sites thereon. Such alternative configurations are within the scope of the present invention.

Protrusions 18, 20 and 22 may be configured to provide advantages with respect to attachment of seal member 12 to the organ. Protrusions 18, 20 and 22 may enable seal member 12 to more easily conform to the irregular shape of the organ, which may aid the formation of a good seal. Protrusions 18, 20 and 22 may access sites on the organ that are most conducive to the forming a good seal with distal edge 14. Protrusions 18, 20 and 22 may accommodate patient-to-patient variations in the size and shape of organs.

Edge 14 provides the primary surface area of contact with the organ and is the surface upon which the force created by the action of the vacuum source acts. By increasing the length of edge 14, protrusions 18, 20 and 22 may increase the surface area of the seal, increasing the surface area upon which the force acts. Increasing the surface area of the seal increases the force applied to the organ for a fixed level of power of the vacuum device, increasing the strength of the attachment between seal member 12 and the organ.

Increased strength of attachment is advantageous. When grasping or lifting an organ, it is important to provide a robust and stable grip for the duration of a particular surgical procedure. The duration of procedures could range, for example, from 10 to 30 minutes. In the case of a beating human heart, the weight to be held could typically range from 1.5 to 3.5 pounds. Because dropping the heart during an anastomotic procedure could cause severe damage to the artery being repaired, it is important to hold such weight with a reasonable margin for safety.

Increasing the area upon which the vacuum force acts may also reduce the likelihood of damage to the surface of the organ, such as abrasion, laceration, or perforation. Further, protrusions 18, 20 and 22 may expose areas of the organ for surgical procedures that require access to these areas of the organ.

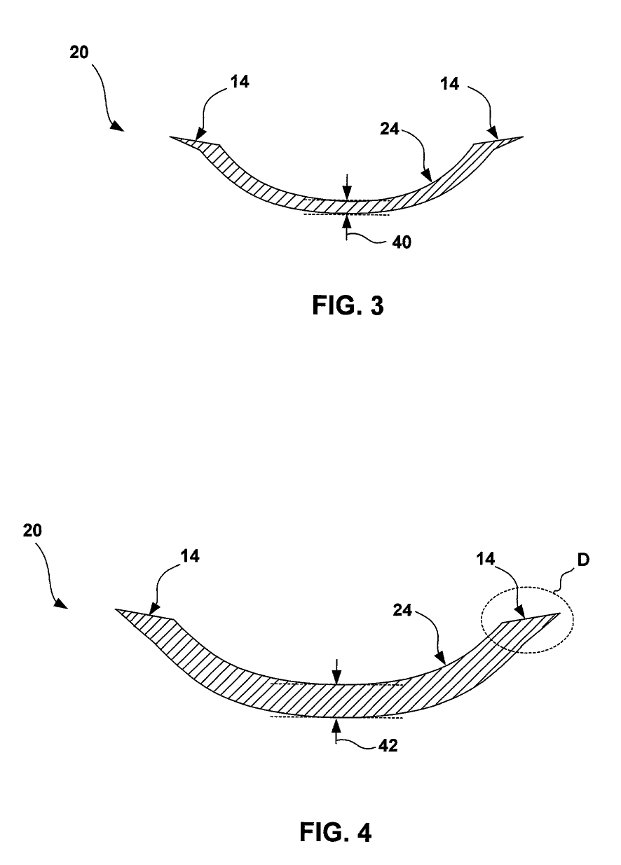

FIG. 3 is a cross-sectional view of one protrusion, the cross-section taken at line A indicated in FIGS. 1 and 2. FIG. 4 is a cross-sectional view of one protrusion, the cross-section taken at line B indicated in FIGS. 1 and 2. In both of FIGS. 3 and 4, edge 14 and inner surface 24 are visible. The relatively flat surface of edge 14 facilitates contact between edge 14 and the organ over the substantially all of the surface area of edge 14. This in turn facilitates the formation of the seal between edge 14 and the organ. The concavity or curvature of protrusion 20, and of seal member 12 in general, further facilitates contact between edge 14 and the organ over the substantially all of the surface area of edge 14, and facilitates the definition of the chamber when seal member 12 is engaged with the surface of the organ.

The wall thickness of seal member 12 near its center is generally greater than the wall thickness at the distal ends of protrusions 18, 20 and 22. This distribution of the mass of seal member 12 may be advantageous. A greater percentage of the weight of the organ is supported near the center of seal member 12, thus a comparatively thick wall in that area may be desired. Near the distal ends of protrusions 18, 20 and 22, less weight is supported, and more flexibility may be desired. Thus, a comparatively thin wall in that area may be desired.

Thickness 40, as shown in FIG. 3, is a thickness of protrusion 20 at point A indicated in FIGS. 1 and 2. Thickness 42, as shown in FIG. 4, is a thickness of protrusion 20 at point B indicated in FIGS. 1 and 2. The difference between thickness 40 and thickness 42 illustrates this advantageous distribution. The present invention, however, is not limited to such a distribution, but instead covers any distribution of the mass of seal member 12. For example, a seal member 12 of substantially constant thickness could be employed.

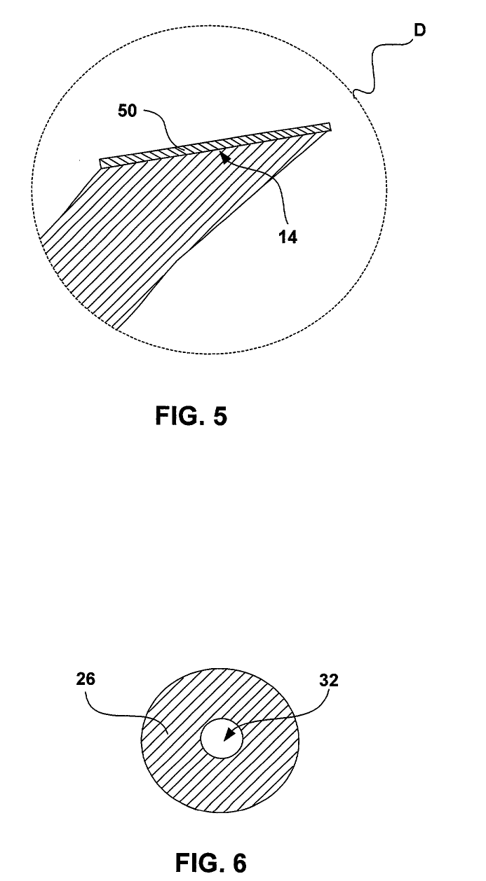

FIG. 5 is an enlargement of area D in FIG. 4 showing distal edge 14 of seal member 12. As shown in FIG. 5, edge 14 can be optionally coated with a coating 50 that is substantially more compliant and compressible than edge 14 and seal member 12. Coating 50 may be tacky, which may assist in the adhesion of seal member 12 to the slippery surface of an organ. Coating 50 may aid in the formation of the seal between edge 14 and the organ. Coating 50 may also reduce the likelihood of damage to the surface of the organ, such as abrasion, laceration, or perforation. Coating 50 may be included at every point on distal edge 14, or at selected points.

Coating 50 could, for example, include a silicone or other polymer of a lower durometer than edge 14 and seal member 12. Coating 50 could, for example, consist of a silicone gel, hydrogel or closed cell foam. Use of Shore A 5-10 durometer silicone elastomer for the coating 50 may be appropriate for some applications. Silicone gels may be preferred, however, due to the intrinsic compliance and tackiness provided by such materials.

Like silicone elastomers, silicone gels can be manufactured with a range of crosslink densities. Silicone gels, however, do not contain reinforcing filler and therefore have a much higher degree of malleability and conformability to desired surfaces. As a result, the compliance and tackiness of silicone gel materials can be exploited in coating 50 to provide a more effective seal.

Examples of suitable silicone gel materials are MED-6340 and GEL-8150, both commercially available from NUSIL Silicone Technologies, of Carpinteria, Calif. The MED-6340 silicone gel is tacky and exhibits a penetration characteristic such that a 19.5 gram shaft with a 6.35 mm diameter has been observed to penetrate the gel approximately 5 mm in approximately 5 seconds. This penetration characteristic is not a requirement, but merely representative of that exhibited by the commercially available MED-6340 material.

Each gel is provided as a two-part component liquid, the components designated Part A and Part B, which may be blended together. In general, increasing the ratio of Part A to Part B produces a softer and tackier gel, while increasing the ratio of Part B to Part A produces a firmer less tacky gel. When MED-6340 is mixed in a A:B ratio of approximately 1:1, the resulting silicone gel is suitable for use as coating 50. The material adheres well to slippery organs, such as the heart, and is also easily moldable. In addition, the material minimizes tissue abrasion and poses virtually no risk of trauma to the heart.

FIG. 6 is a cross-sectional view of nipple 26 of manipulation device 10, the cross-section taken at line C indicated in FIG. 1. Passage 32 is visible. Nipple 26 is robust enough to support the weight of the organ and distribute lifting or pulling forces to seal member 12 and protrusions 18, 20 and 22. In order to provide this robustness, nipple 26 may be relatively thick-walled. The thick walls of nipple 26 may also provide structural support for a handle or other fixed support.

Nipple 26 may also be constructed to be flexible, permitting side-to-side motion, oscillatory motion around the long axis of nipple 26, and up and down motion along the long axis of nipple 26. Nipple 26 may be made long and flexible to bend without kinking and closing passage 32. Thus, nipple 26 may be manipulated, or a handle or support may be attached thereto, without adversely affecting passage 32. In embodiments where seal member 12 is to be adhered to a beating heart, the flexibility of nipple 26 may permit the beating motion of the heart to occur without loss of seal and adhesion. The flexibility of nipple 26 is particularly advantageous in these situations, because loss of seal and adhesion may, in some circumstances, allow the heart to drop back into the chest, which may cause trauma to the heart, and may interfere with the progress of the operation.

In some embodiments of the present invention nipple 26 may be located in the center of seal member 12. Because of the central location of nipple 26 in such embodiments, upward force applied to nipple 26 is fairly uniformly distributed to seal member 12 and protrusions 18, 20 and 22. In embodiments where seal member 12 is to be adhered to a peri-apical site of a beating heart, the central location of nipple 26 provides a further advantage by permitting a surgeon or assistant to lift the heart from the apical region while keeping tension on the long axis of the heart. The tension prevents bending of the long axis, which prevents distortion of valves and the decline in cardiac output that occurs when the heart is lifted by the surgeon's hand alone.

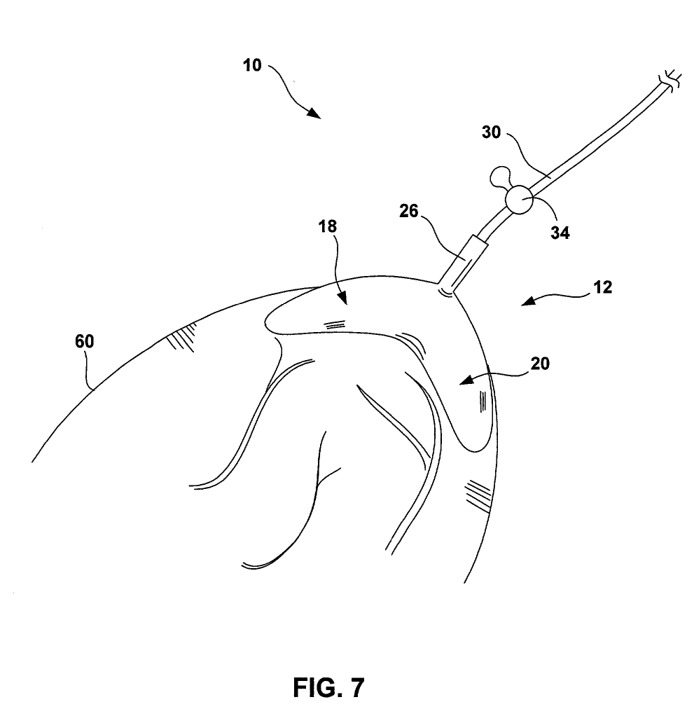

FIG. 7 is a perspective view of one method of deployment of the organ manipulation device 10 shown in FIG. 1, with the seal member 12 and protrusions 18, 20 and 22 on the apex of a human heart 60. Protrusions 18 and 20 are visible, while protrusion 22 is obscured by heart 60. Seal member 12 is connected by nipple 26 to vacuum tube 30, which provides suction from the vacuum source to adhere seal member 12 to heart 60 as described above.

A person, such as a surgeon or assistant, may position seal member 12 on the heart before applying suction from the vacuum source to adhere seal member 12 to heart 60. Using a device with a seal member with two or more protrusions permits positioning the device on heart 60 in a position such that it conforms to asymmetric and irregular surfaces, including such features as fat deposits. The person can manually position seal member 12 to achieve optimal sealing under vacuum prior to lifting or manipulating device 10 and heart 60. The person can further position seal member 12 so as not to obscure important structures on heart 60, such as the coronary arteries. The person can further position seal member 12 so as not to interfere with the hemodynamics of heart 60 when manipulating heart 60, if heart 60 is beating.

The person may apply vacuum pressure to adhere seal member 12 to heart 60 by any of the means described above. When seal member 12 is adhered to heart 60, nipple 26, or a handle or fixed support affixed thereto, may be used to lift or support heart 60.

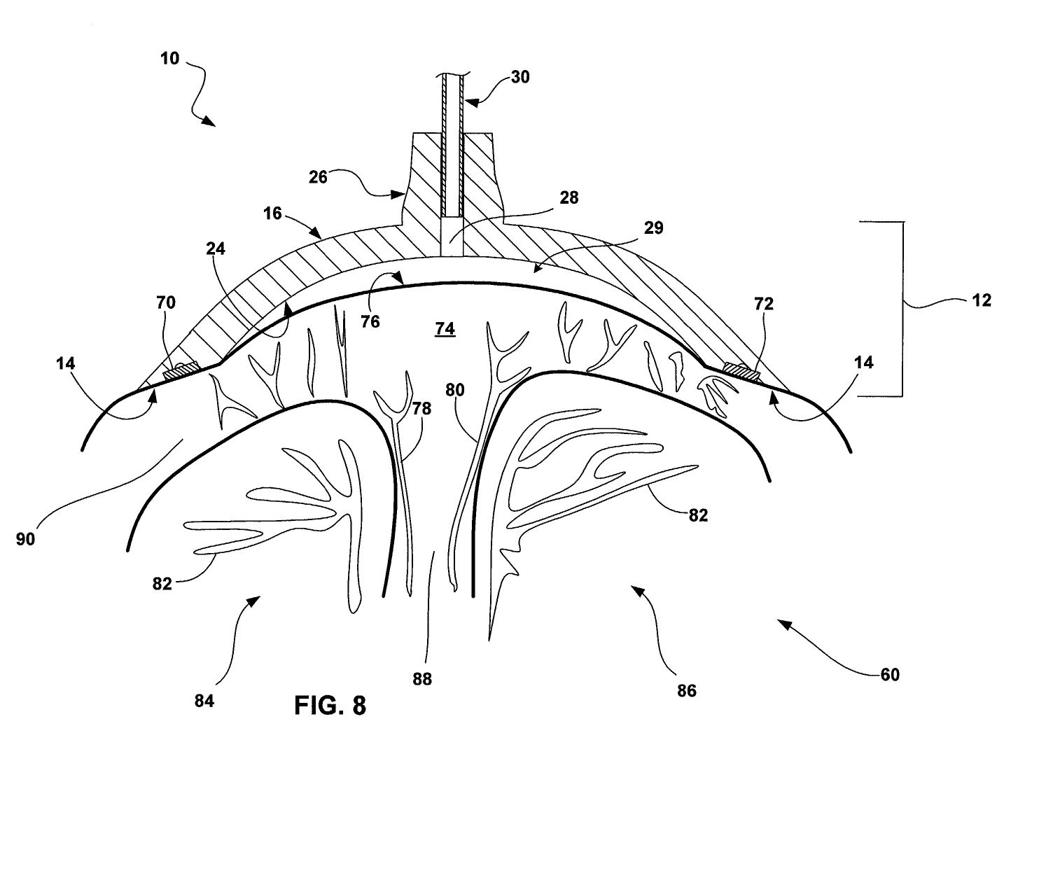

In some alternative embodiments of the present invention, organ manipulation device 10 may also be used to pace heart 60. FIG. 8 is a cross-sectional side view of such an embodiment of device 10, including seal member 12 and electrodes 70 and 72 incorporated within seal member 12 that deliver pacing pulses to heart 60. Seal member 12 is shown deployed on the apex 74 of the heart 60. In the embodiment depicted in FIG. 8, electrodes 70 and 72 are located within distal edge 14 of seal member 12.

Electrodes 70 and 72 may be located anywhere within or proximate to distal edge 14, central body 16 or protrusions 18, 20 and 22 (not visible in FIG. 8), as long as contact between electrodes 70 and 72 and the surface 76 of heart 60 is possible. There may be little contact, for example, where chamber 29 is defined by seal member 12 and heart 60. Contact between electrodes 70 and 72 and surface 76 may be most likely to occur when electrodes 70 and 72 are located within or proximate to distal edge 14. Surface 76 may be the pericardial sac of heart 60, or the epicardium of heart 60 if the pericardial sac is opened.

Electrodes 70 and 72 may be used to pace heart 60 by stimulation of the bundles of His 78 and 80, and Purkinje fibers 82. The normal pacemaker of heart 60 is the sinoatrial (SA) node (not shown in FIG. 8). The SA node is a small specialized region in the right atrial wall near the opening of the superior vena cava. An action potential initiated within the SA node ordinarily spreads to both atria of heart 60. An internodal pathway extends from the SA node to the atrioventricular (AV) node (not shown in FIG. 8), which is a small bundle of specialized cardiac muscle cells near the junction of the atria and the ventricles 84 and 86. Specialized cells known as the bundle of His extend from the AV node, through the ventricular septum 88, where they divide into the left branch bundle of His 78 and the right branch bundle of His 80. The branch bundles of His 78 and 80 curve around the tips of ventricular chambers 84 and 86, and travel back toward the atria along the outer walls of heart 60.

Following receipt of an impulse by the AV node from the SA node, and after a brief AV nodal delay, the impulse travels rapidly down the bundles of His 78 and 80. Purkinje Fibers 82 extend from bundles of His 78 and 80 and spread throughout the ventricular myocardium 90. The impulse transmitted by the bundles of His 78 and 80 is carried throughout the ventricular myocardium 90 by Purkinje fibers 82. The bundles of His 78 and 80 and Purkinje fibers 82 have a normal rate of action potential discharge of 20 to 40 action potentials per minute.

Stimulation of bundles of His 78 and 80 and Purkinje fibers 82 may cause the ventricular myocardium to beat at a faster rate and the help pace heart 60. Electrodes 70 and 72, which may be coupled to a voltage or current source (not shown in FIG. 8) via conductors, may in this way be used to stimulate the bundles of His 78 and 80 and Purkinje fibers 82 and help pace heart 60.

Any number of electrodes may be included within seal member 12, and used to pace heart 60. Because seal member 12 adheres atraumatically to apex 74, device 10 can remain on apex 74 for long periods of time without causing hematoma or other trauma. In addition, the placement of seal member 12 on apex 74 allows for minimal interference with the surgical field. Consequently, device 10 can pace heart 60 when needed, and can remain in place when pacing is not required.

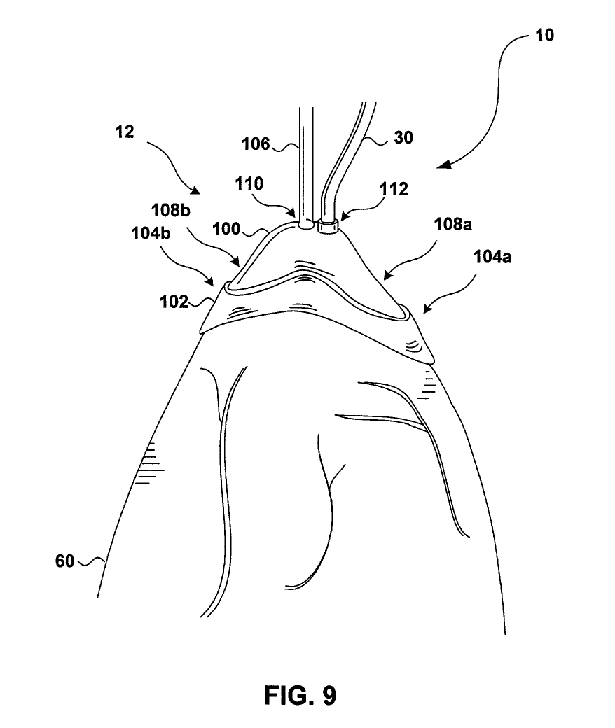

FIG. 9 is a perspective view of another alternate embodiment of the organ manipulation device 10 deployed on the apex of heart 60. In this embodiment, seal member 12 comprises a central member 100 and a distal skirt-like member 102.

Central member 100 is connected to vacuum tube 30. In this embodiment, central member 100 is also connected to a dedicated support shaft 106, which may support most or all of the weight of the organ. Vacuum tube 30 may provide little or no load-bearing capability. Support shaft 106 may provide no vacuum pressure to manipulation device 10.

Support shaft 106 and vacuum tube 30 need not be coupled to central member 100 in the same way. In FIG. 9, support shaft 106 is coupled to central member 100 with a swivel connection 110. Vacuum tube 30 is flexible and is coupled to central member 100 with a fixed connection 112. Swivel connection 110 and the flexibility of vacuum tube 30 cooperate to accommodate the beating motion of heart 60 while maintaining a good seal and good adhesion.

In the embodiment shown in FIG. 9, both central member 100 and skirt-like member 102 cooperate to define a plurality of protrusions 104a, 104b (hereinafter collectively 104) that extend radially outward from the center of seal member 12. Protruding structures 108a, 108b (hereinafter collectively 108) extend outward from the center of central member 100, and protrusions 104 extend from the protruding structures 108. Protruding structures 108 may enhance the structural integrity of protrusions 104 by, for example, causing protrusions 104 to be oriented in a particular direction.

As shown in FIG. 9, protrusions 104 need not be of uniform size or shape. Protrusion 104a, for example, may extend further outward from the center of seal member 12 than protrusion 104b. Similarly protruding structures 108 need not be of uniform size or shape. Protruding structure 108a may extend further outward from the center of central member 100 than protruding structure 108b. Manipulation device 10 may include additional protrusions and protruding structures that are not shown in FIG. 9. Manipulation device 10 may include, for example, two other protrusions and protruding structures that are not visible in FIG. 9. The protrusions and protruding structures may be, but need not be, sized or shaped similarly to protrusions 104 and protruding structure 108 that are depicted in FIG. 9.

The invention is not limited to any particular shape of central member 100, however. In some embodiments, skirt-like member 102 may alone define protrusions 104, while central member 100 includes no protruding structures. Central member 100 may have, for example, a cup-like shape.

Central member 100 may be formed from many materials, including thermoplastic such as polycarbonate, ABS, polysulfone, polyester and polyurethane, and including corrosion-resistant metals such as titanium, and including rigid and semi-rigid elestomers such as a silicone rubber, natural rubber, synthetic rubber, and polyurethane. Central member 100 may have a semi-rigid structure that may be somewhat compliant, but generally resistant to deformation. As an example, central member 100 may be formed from silicone elastomers in the range of Shore A 30 to 75 durometer.

Skirt-like member 102, in contrast, may be formed from a substantially compliant material, such as a silicone elastomer, silicone gel, hydrogel, closed cell foam or combinations thereof. Skirt-like member 102 generally permits deformation upon contact with tissue. In this manner, central member 100 imparts structural integrity to manipulation device 10, while skirt-like member 102 provides a seal interface with the tissue of an organ, such as heart 60. Also, the material forming skirt-like member 102 may be tacky, and thereby promote adhesion to the surface of the organ. Use of Shore A 5-10 durometer silicone elastomer for the skirt-like member 102 may be appropriate for some applications. Silicone gels may be preferred, however, due to the intrinsic compliance and tackiness provided by such materials. MED-6340 and GEL-8150 are examples of suitable silicone gels that may be employed to form skirt-like member 102.

The invention can provide one or more advantages. For example, protrusions may enable a seal member to more easily conform to the irregular shape of the organ, which may aid the formation of a good seal between the seal member and the surface of the organ. In addition, the protrusions may help the manipulation device adhere to sites on the organ that are most conducive to the forming a good seal with the distal edge and may accommodate patient-to-patient variations in the size and shape of organs. Protrusions may also accommodate motion of the organ without a loss of the seal or adhesion.

Further, the protrusions may increase the surface area of the seal between the distal edge and the surface of the organ. An increased surface area may increase the strength and robustness of the adherence between the seal member and the organ. An increased surface area may also reduce the likelihood of damage to the surface of the organ, such as injury due to abrasion, laceration, or perforation.

Protrusions may also expose areas of the organ for surgical procedures that require access to these areas of the organ. When placed proximate to the apex of the heart, for example, the manipulation device may be oriented to expose a desired region near the apex. Furthermore, the protrusions may be configured with a variety of shapes to allow attachment of a manipulation device to a variety of sites on a variety of organs for a variety of surgical procedures.

The seal member may be made materials that are substantially compliant such that the seal member substantially conforms to the surface of an organ, which in turn promotes formation of a substantial seal between the seal member and the surface of the organ. The seal member and protrusions may exhibit a concave shape, which may also promote the formation of the substantial seal. The distal edge of the seal member may be coated with a coating, such as silicone gel, which may be tacky. The coating may further promote formation of a substantial seal, and may reduce the likelihood of damage to the surface of the organ.

Where a vacuum tube or a dedicated support member is attached to the manipulation device by a nipple that is flexible, the flexibility of the nipple may accommodate the beating motion of a heart without loss of seal and adhesion. The nipple may provide a convenient attachment for a handle, which allows a surgeon or assistant to manipulate an organ, or a fixed support, which may be used to hold an organ in place. In embodiments where the nipple is located near the center of the seal member, the central location permits a surgeon or assistant to lift a heart from the apical region while keeping tension on the long axis of the heart. The tension prevents bending of the long axis, which prevents distortion of valves and the decline in cardiac output that occurs when the heart is lifted by the surgeon's hand alone.

Various embodiments of the invention have been described. These embodiments are illustrative of the practice of the invention. Various modifications may be made without departing from the scope of the claims. For example, the invention encompasses manipulation devices having protrusions of a variety of shapes, sizes and positions. Although FIGS. 1 and 2, for example, depict a seal member including three protrusions, the number or protrusions may be varied. Moreover, the angle or distance between protrusions, and the length, width and shape of each protrusion may also be varied. These variations may allow attachment of the manipulation device to a variety of sites on a variety of organs for a variety of surgical procedures.

One example of an alternative configuration may include two protrusions extending in opposite directions from the central body, with the protrusions increasing in width towards their distal ends. In this configuration, the device may resemble a bowtie or a propeller. Another example of an alternative configuration may include two protrusions of approximately equal length separated by an angle of 180 degrees, and two substantially shorter protrusions, each equidistant from the two longer ones. The invention encompasses all of these variations.

The seal member need not be made of silicone, and need not be flexible. Nor is the seal member limited to a generally concave shape. As illustrated by FIG. 9, the seal member is not limited to single-piece construction, but may instead comprise two or more components.

The distal edge shown in the figures is exemplary. The distal edge need not be substantially flat, but may be shaped with a topography that enhances attachment to a particular organ or site. The distal edge or the coating on the device need not be smooth, but may be textured to enhance attachment to an organ.

The vacuum tube may be load-bearing or non-load-bearing. The vacuum tube need not be flexible. For example, the vacuum tube may comprise a segmented hollow articulable arm that allows manipulation and positioning of an organ and may be locked to provide support for the organ in a position. Coupling of a manipulation device to any type of vacuum tube is within the scope of the present invention.

In some embodiments of the invention, Moreover, the manipulation device need not be coupled to a vacuum tube at all. A surgeon may move the organ and/or the seal member to displace air from the chamber defined when the seal member is in contact with the surface of the organ. In some circumstances, displacement of air may be sufficient to create a seal between the device and the organ.

The nipple as depicted in FIGS. 1, 7 and 8 is merely exemplary, as are the flexible and fixed connections shown in FIG. 9. The invention is not limited to the particular connections shown. In addition, the invention encompasses embodiments in which a flexible joint or swivel connection is a part of a vacuum tube or a support shaft, or both. The invention also encompasses embodiments in which a flexible connector is an integral part of the central body.

Moreover, features from some embodiments described herein may be incorporated into other embodiments. For example, the manipulation device depicted in FIG. 1 may be adapted to include the multi-piece construction shown in FIG. 9.

Furthermore, the manipulation device is not limited to attachment to a peri-apical site of a heart, nor is the manipulation device limited to use during CABG. Rather, the various embodiments of the manipulation device may be used in any surgical procedure that may involve manipulation of an internal organ.

These and other embodiments are within the scope of the scope of the following claims.

1. A device comprising:
a seal member to contact a surface of an organ, the seal member including an inner surface, a central body, and at least two protrusions that extend outward from the central body.
wherein the inner surface defines a chamber when the seal member is in contact with the surface of the organ.

2. The device of claim 1, wherein the seal member includes at least three protrusions.

3. The device of claim 1, wherein the protrusions are located equidistant from each other.

4. The device of claim 1, wherein the protrusions are of equal size and shape.

5. The device of claim 1, wherein the protrusions exhibit a long and narrow geometry.

6. The device of claim 1, wherein each protrusion includes a proximal end and a distal end, and wherein the width of a protrusion tapers from the proximal end to the distal end.

7. The device of claim 1, wherein each protrusion includes a proximal end and a distal end, and wherein the thickness of a protrusion tapers from the proximal end to the distal end.

8. The device of claim 1, wherein the protrusions are substantially flexible and compliant.

9. The device of claim 1, wherein the seal member is formed from silicone.

10. The device of claim 1, wherein the seal member includes a distal edge that extends around the perimeter of the seal member and contacts a surface of the organ.

11. The device of claim 10, further comprising a coating located on the distal edge.

12. The device of claim 11, wherein the coating is tacky.

13. The device of claim 11, wherein the coating comprises silicone gel.

14. The device of claim 1, wherein the seal member further includes a vacuum port in fluid communication with the chamber.

15. The device of claim 14, further comprising a valve to regulate fluid flow through the vacuum port.

16. The device of claim 1, further comprising:
at least one of a vacuum tube and a support shaft; and
a connector connected to at least one of the vacuum tube and support.

17. The device of claim 16, wherein the connector is located on a proximal end of the seal member.

18. The device of claim 16, wherein the connector is formed integrally with the seal member.

19. The device of claim 16, wherein the connector is flexible.

20. The device of claim 16, wherein the seal member further includes a vacuum port in fluid communication with the chamber, and
wherein the connector is in fluid communication with the vacuum tube and the vacuum port.

21. The device of claim 16, further comprising a handle, the handle attached to at least one of the seal member, the connector, the tube and the support shaft.

22. The device of claim 16, further comprising a fixed support that holds the organ in a position, the fixed support attached to at least one of the seal member, the connector, the tube and the support shaft.

23. The device of claim 1, further comprising an electrode affixed to the seal member.

24. The device of claim 23, wherein the electrode is located on the distal edge.

25. The device of claim 1, wherein the seal member comprises a central member and a skirt-like member.

26. The device of claim 25, wherein the skirt-like member is substantially compliant and tacky, and
wherein central member is less deformable than the skirt-like member, thereby imparting structural integrity to the seal member.

27. A device comprising:
a seal member to contact a surface of an organ, the seal member including a central member and skirt-like member, wherein the skirt-like member includes at least two protrusions that extend outward from the central member.

28. The device of claim 27, wherein the skirt-like member is substantially compliant and tacky.

29. The device of claim 27, wherein central member is less deformable than the skirt-like member, thereby imparting structural integrity to the seal member.

30. The device of claim 27, wherein the seal member includes an inner surface that defines a chamber when the seal member is in contact with the surface of the organ.

31. The device of claim 30, wherein the seal member further includes a vacuum port in fluid communication with the chamber.

32. The device of claim 27, wherein the skirt-like member further includes a distal edge extending around the perimeter of the skirt-like member.

33. The device of claim 32, further comprising a coating located on the distal edge that is more compliant and tacky than the skirt-like member.

34. The device of claim 27, further comprising at least one of a handle and a support affixed to the device configured to permit the device to be manipulated and to be immobilized relative to a body of a patient.

35. The device of claim 27, wherein the central member includes a protruding structure that extends outward from the center of the central member, wherein at least one protrusion extends from the protruding structure.

36. The device of claim 27, wherein the central member has a cup-like shape.

37. A method comprising:
bringing a seal member into contact with a surface of an organ to define a chamber, the seal member having at least two protrusions;
bringing the protrusions into contact with the surface of the organ; and
applying vacuum pressure to the chamber to adhere the seal member and the protrusions to the surface.

38. The method of claim 37, wherein applying vacuum pressure comprises causing at least one of the protrusions to deform slightly.

39. The method of claim 37, wherein applying vacuum pressure comprises forming a seal between the seal member and the surface of the organ.

40. The method of claim 37, wherein applying vacuum pressure comprises closing a valve to maintain a first fluid pressure within the chamber at a lower value than a second fluid pressure outside of the seal member.

41. The method of claim 37, further comprising manipulating the organ by manipulating the seal member.

42. The method of claim 41, wherein manipulating the organ comprises manipulating the organ by manipulating a handle attached to the seal member.

43. The method of claim 42, further comprising:
moving the organ from a first position to a second position; and
maintaining the organ in the second position by attaching the seal member to a support.

44. The method of claim 37, wherein the organ is a heart, further comprising delivering pacing pulses to the heart.
